(19) United States
(12) Patent Application Publication
(10) Pub. No.: US 2018/0348188 A1
(43) Pub. Date: Dec. 6, 2018

(54) METHOD OF USING BIOMOLECULES TO PREDICT SOURING IN OILFIELD SYSTEMS

(71) Applicant: Dow Global Technologies LLC, Midland, MI (US)

(72) Inventors: Brandon Morris, Zurich (CH); Geert Van Der Kraan, Lachen (CH)

(21) Appl. No.: 15/991,072

(22) Filed: May 29, 2018

Related U.S. Application Data
(60) Provisional application No. 62/515,098, filed on Jun. 5, 2017.

(51) Int. Cl. G01N 33/18; G01N 33/24

(52) U.S. Cl. CPC G01N 33/1826; C02F 1/50; G01N 33/24

(57) ABSTRACT

The present invention is a method for the early detection or prediction of souring of aqueous systems by detecting a population of lipid and organic acids and the treatment of such systems.

Currently, the production of oil and gas is becoming increasingly more difficult as secondary recovery techniques are exploited to displace the oil in the reservoirs and maintain reservoir pressure. Many oilfield operations occur with fluids (generally referred to as “wellbore fluids”) that circulate through or are otherwise introduced into the borehole. These fluids may include drilling fluids that are injected and circulated during the drilling of the well, well stimulation fluids, completion fluids that may be circulated during or after drilling during various completion operations, and fracturing fluids which may be used after drilling in order to stimulate the well to increase production from a hydrocarbon reservoir. The fluids are generally water, often sea water or brine, and can also contain high viscosity fluid additives or friction reducers. As oilfields become more mature, the water production from oil producing wells increases.

The different water streams must be separated from the oil, using topside facilities (separators, deaerators, pipes, etc.), which often represent a considerable value to a company. The oil which is separated from the water for further refinement is another source of expense. With the introduction of water, (either from the formation or introduced via seawater), microbial contamination is also a problem. Microbial contaminants, particularly bacteria and archaea, can grow and proliferate on the surface or downhole of the well and on drilling and pipeline surfaces.

Most of these oil and gas environments, such as wells, tanks, pipelines and separators are anoxic, elevated in temperature and highly saline. Nitrate addition may take place in parts of the system; and other parts of industrial systems can be aerobic. In facilities or industrial units that serve oil fields (wells, turrets, pipes, separators, etc.), the environment can change dramatically, driven by the amended water chemistry in different elements of the industrial units, the availability of a specific electron acceptor, and the changes of the environmental parameters. Environmental factors, such as high salinities and temperatures, metal pollution, etc. turn oil and gas environments into ecosystems that encourage microbial growth.

One of the most well-known problems due to microbial contamination includes the formation of H₂S via microbial respiration of sulfate (souring). The formation of H₂S via sulfate reduction is often initiated via the introduction of sulfate via seawater injection (the average sulfate concentration in seawater is 2.72 g/kg). Other problems include the degradation of functional chemicals such as corrosion inhibitors, oxygen scavengers, and demulsifiers and biofilm formation that may enhance corrosion and/or hinder proper functioning of equipment (e.g., clogging of filters and valves, decrease heat transfer, etc.).

The biogenic production of H₂S is especially problematic for oil and gas wells because H₂S is highly toxic to humans. The biogenic production of H₂S presents a substantial risk for people working in the industry. Souring also causes degradation of the oil leading to the need for desulfurization of oil, which presents a significant cost to oil refinement. Hydrogen sulfide also reacts with metal surfaces causing corrosion, which affects asset integrity of storage tanks, pipelines, valves, etc. This accelerated localized corrosion of metal surfaces which is caused by the metabolic activity of microorganisms often takes place under a biofilm or other deposit types and puts the physical assets at risk of failure much sooner than their expected and budgeted lifetimes. This has a significant impact on the cost management of above ground facilities. Additionally, increased H₂S levels cause quality degradation of the hydrocarbons as it must be removed again at a later stage (desulfurization). This also has a cost impact on the hydrocarbons eroding the already small margins of the business. The production of H₂S and scale deposits in the reservoir as a consequence can cause conformance issues of the reservoir itself (e.g. hindrance of phase flow gas, oil, water, reservoir or the subsurface strata where the hydrocarbons are produced). Souring remains difficult to assess in terms of severity and is even more difficult to detect before it becomes problematic.

Energy and associated service companies often revert to microbial control programs in order to reduce the amounts of microorganisms that grow in the different water phases and exert these activities. Biocides and antimicrobials may be used to inhibit and/or remove microbial growth in the water. The presence of biocides in the water, which must be disposed of or recycled back into seawater itself poses an additional problem for the industry. However, it remains difficult to assess the severity of the problem presented by the presence of microorganisms thriving in oil (gas)-water systems as most of these reservoirs and industrial systems cannot be ‘opened’ and are designed to be ‘closed’ systems with limited sample points.

Because many organisms are unculturable, typical microbiology analyses in the petroleum industry include cell density checks determining how many cells are present in the wells, e.g. cells/ml, and ecological studies. While biocide compositions are available that provide adequate biocidal activity in downhole operations, the testing of the oilfield fluids will conventionally take weeks to provide information regarding the efficacy of the biocide. The efficacy of the biocide (or any treatment process) may be affected by the biocide composition, the dosing amount of the biocide, and outside factors such as, but not limited to, chemical and/or sand compositions used in the wellbore composition.

Thus, there is a need for improved technologies which allow the early detection of souring caused by uncontrolled growth of microorganisms in oil and gas facilities. This would enable monitoring of remediation processes in the field and allow operators to detect souring earlier in the life of a field.

The present invention provides a method for the early detection of souring comprising preparing an aqueous sample comprising microorganisms; and, analyzing the sample for the presence of lipids and organic acids selected from the group consisting of iso- and anteiso-methyl branched fatty acids containing 13, 15, or 17 carbons, iso-methyl branched fatty acids containing 18 carbons, 3,4-dimethylbenzoic acid, 3,5-dimethylbenzoic acid, and 3-ethylbenzoic acid; wherein the presence of the combination of one or more of 3,4-dimethylbenzoic acid, 3,5-dimethylbenzoic acid, and 3-ethylbenzoic acid at a concentration greater than or equal to 0.8% combined with four or more lipids indicates the presence of souring.

The present invention further provides a method for treating a souring aqueous system comprising detecting souring according to the method of claim 1; and applying a biocide, hydrogen sulfide scavenger, or combination thereof.

Technical and scientific terms used herein have the same meaning as commonly understood by one of ordinary skill in the art to which this invention belongs and by reference to published texts, which provide one skilled in the art with a general guide to many of the terms used in the present application. Any definitions are provided for clarity only and are not intended to limit the claimed invention. Unless otherwise specified herein, % are weight % based upon weight of total composition.

Aqueous systems of the present invention include any aqueous sample that is susceptible to microbial attack of microorganisms. Suitable aqueous systems include oil and gas application injection waters as well as waters produced by oil and gas operations.

Microorganisms include but are not limited to bacteria and in particular, sulfate reducing bacteria. The aqueous systems are then analyzed using techniques known to the art for detecting lipids and organic acids. Lipids relevant to the present invention are iso- and anteiso-methyl branched fatty acids containing 13, 15, or 17 carbons, iso-methyl branched fatty acids containing 18 carbons. Once four or more lipids are detected in combination with greater than or equal to 0.8% of relevant organic acids of the present invention, a well/aqueous system is considered to be soured or such fact is indicative that souring will occur. Organic acids of interest in the present invention are 3,4-dimethylbenzoic acid, 3,5-dimethylbenzoic acid, and 3-ethylbenzoic acid. In one embodiment, all 3 organic acids are present in a collective concentration of greater than or equal to 0.8%. In another embodiment, each of 3,4-dimethylbenzoic acid, 3,5-dimethylbenzoic acid, and 3-ethylbenzoic acid is present in a concentration of greater than or equal to 0.8%.

Once there is an indication of souring or after souring has occurred, the aqueous system may be treated with at least one biocide, at least one hydrogen sulfide scavenger, and.or a mixture thereof. When the aqueous system is an oilfied water, treatment includes the application of such materials either topside or downhole by known methods of delivering materials to an oilfield.

### EXAMPLES

### Extraction of Organic Acids

The samples collected for metabolite analysis consisted of 1 L of water, either taken directly from a tank or separated from the oil. After which, they were alkalized using 50 ml of a saturated NaHCO₃-solution. The organic acids were then extracted three times with 300 ml of chloroform. Subsequently, the aqueous phase was acidified using 5 ml HCl (12 M) and extracted with 300 ml of ethyl acetate. The organic phases were combined, dried over sodium sulfate and evaporated to dryness under a stream of nitrogen. The extract was re-dissolved in 1 ml of diethyl ether for derivatization. Phenylsuccinic acid (100 μg) was added as an internal standard, to allow quantification of the detected acids.

### Derivatization

Half of the organic acid extract was used for methylation with diazomethane and analyzed by GC-MS. The other half was taken to derivatize succinic acids to succinimides using (R)-1-phenylethanamine. For a detailed description of the latter derivatization technique, see Jarling et al. (2015). Jarling, R., Kühner, S., Basilio Janke, E., Gruner, A., Drozdowska, M., Golding, B. T., Rabus, R., Wilkes, H., 2015. Versatile transformation of hydrocarbons in anaerobic bacteria: substrate ranges and regio- and stereo-chemistry of activation reactions. Frontiers in Microbiology, 6:880.

### Medium-Pressure Liquid Chromatography

Medium-pressure liquid chromatography was performed according to the procedure described by Radke et al. (1980). Radke, M., Willsch, H., Welte, D. H., 1980. Preparative hydrocarbon group type determination by automated medium pressure liquid chromatography. Analytical Chemistry 51, 406-411. The crude oil sample (30 mg) was diluted in 600 μl n-hexane and injected. Hetero compounds were retained on the pre-column. The aliphatic hydrocarbons were eluted with 40 ml n-hexane (flow rate: 8 ml/min). After an inversion of the flow direction, the aromatic hydrocarbons were eluted with 72 ml n-hexane (flow rate: 8 ml/min) from the main column. All fractions were vaporized and dried under a stream of nitrogen.

### GC-MS Analysis

The gas chromatographic-mass spectrometric (GC-MS) analysis was performed on a gas chromatograph equipped with a fused silica capillary column (50 m length; 0.22 mm inner diameter; 0.25 μm film thickness, SGE Analytical Science). A sample volume of 1 μl was injected into the Programmed Temperature Vaporization (PTV) injector in splitless mode. The injector was programmed with a heating rate of 10° C./s from 50° C. to 300° C. Helium was used as a carrier gas with a flow rate of 1 ml/min. The GC oven was programmed to ramp from 50° C. (1 min hold time) to 310° C. (held for 30 min) with a heating rate of 3° C./min. The ion source temperature of the mass spectrometer was 230° C. and the ionization was performed by EI (electron impact) with an ionization energy of 70 eV. Mass spectra were recorded over a range of m/z 50-600 (derivatized organic acids of water extracts) and m/z 50-310 (aromatic hydrocarbon fraction) at a rate of 2.5 scans/s.

### Phospholipid Analysis

Oil-water mixtures from active producing wells were sampled using 10 L jerry cans and allowed to separate into phases before further processing. Water from topside facilities (separator tanks, water buffer tanks, and injectors) was filled into 5 L sterilized glass bottles.

### Extraction of Intact Phospholipids

Directly after sampling, 2 L of each water sample was filtered to collect and concentrate microbial cells using a Satorius filtration system, with glassfibre prefilters in combination 0.2 μm, I.D.: 50 mm filters. The filters containing the microbial cells were treated using a modification of the extraction method described by Bligh and Dyer (1959). Bligh E G, Dyer W J (1959) A rapid method of total lipid extraction and purification. Can J Biochem Phys 37: 911-917.

The filters containing the cells were saturated three times with a solvent mixture of methanol-dichloromethane-ammonium acetate buffer (10 mmol*L−1), 2:1:0.8 (v/v) in a beaker and sonicated for 10 min. The extracts were combined by transfer to a separation funnel and spiked with 50 μg deuterated [D31]-palmitoyl lysophosphatidylcholine (LPC) as an internal standard. The composition of the solvent was changed to about 1:1:0.9 (v/v) by adding dichloromethane and ammonium acetate buffer to allow phase separation. After separation of the organic phase, the water phase was extracted twice with 20 ml dichloromethane. The organic phases were combined, concentrated using a TurboVap® and dried under a stream of nitrogen.

The extract was dissolved in 1 ml of chloroform-methanol, 9:1 (v/v) and applied to a silica gel column (1 g silica gel 63-200 μm) and a florisil column used in sequence and eluted in the following order: 20 ml chloroform (neutral polar fraction), 50 ml methyl formate with 0.025% pure acetic acid (free fatty acid fraction), 20 mL acetone (glycolipid fraction) and 25 ml methanol (phospholipid fraction, only the silica column) and 25 ml of methanol-water, 6:4 (v/v) (highly polar fraction). The latter step contributes to significant improvement in the recovery of highly polar phospholipids from the silica column (Zink K, 2004). Zink K-G, Mangelsdorf K (2004) Efficient and rapid method for extraction of intact phospholipids from sediments combined with molecular structure elucidation using LC-ESI-MS-MS analysis. Anal Bioanal Chem 380: 798-812.

The highly polar fraction was collected and mixed with dichloromethane and water to achieve a methanol-dichloromethane-water ratio of 1:1:09 (v/v). After separation of the organic phase, the water phase was extracted twice with 20 mL dichloromethane. The organic phases obtained from the highly polar fraction were combined with the phospholipid fraction, evaporated and dried under a nitrogen stream.

### Phospholipid Fatty Acid Analysis

Half of the phospholipid-containing extract was used for trans-esterification to obtain PLFAs from the intact phospholipids using a method described in the literature (Müller et al., 1990). Müller K-D, Husmann H, Nalik H P (1990) A new and rapid method for the assay of bacterial fatty acids using high resolution capillary gas chromatography and trimethylsulfonium hydroxide. Zbl Bakt 274: 174-182.

A 500 μl addition of Trimethylsulfonium hydroxide was combined with the extract and the sealed vial was kept at 70° C. for 2 h. A GC-MS analysis was performed using a system equipped with a BPX5 fused silica capillary column (50 m length; 0.22 mm inner diameter; 0.25 μm film thickness). A sample volume of 1 μl was injected into the Programmed Temperature Vaporization (PTV) injector operated in splitless mode. The injector was programmed to ramp from 50° C. to 300° C. (10 min hold time) at a heating rate of 10° C./s. Helium was used as the carrier gas at a flow rate of 1 ml/min. The GC temperature program commenced at 50° C. (held for 1 min) followed by a temperature increase at a rate of 3° C./min to 310° C. (held for 30 min). The ion source temperature of the mass spectrometer was 230° C. The ionization was conducted by electron impact (EI) using an ionization energy of 70 eV. Mass spectra were recorded over a range of m/z 50-600 at a rate of 2.5 scans/s. PLFAs were identified by comparison of the mass spectra and retention times of their methyl esters to reference standards.

Methods of Early Detection of, and/or Prediction of, Souring

The methods and compositions described herein are based upon the discovery that the presence of certain lipids and organic acids of microbial origin can be used as accurate predictors of pending or ongoing souring-related processes. The methods and compositions described herein enable early detection of souring through detection of these specified compounds and permit treatment programs to be designed and implemented for eliminating or minimizing microbial activities to prevent or control souring.

The environments of soured wells, wells in the early process of souring, and non-soured wells can be discriminated from each other based on distinct differences in the presence of indicator lipids and organics acids. Based on geochemical ecosystem parameters and if the wells are suffering from an issue that correlates to the presence and activities of microbes, their environments become more discriminative from each other and results in differences in concentration or lipids and organic acids in wellbore fluids found in the waters from these environments. Qualitative and/or quantitative detection of iso- and anteiso-methyl branched fatty acids containing 13, 15, 17 carbons, or iso-methyl branched fatty acids containing 18 carbons, as well as the organic acids 3,4-dimethylbenzoic acid, 3,5-dimethylbenzoic acid, and 3-ethylbenzoic acid may be used to detect wells or tanks that will sour.

The above described methods may be used on-site at oil or gas processing sites.

One of skill in the art may construct a number of suitable reagent compositions for use in the methods described herein given the teachings of this specification.

In order to further understand the above-described invention and to demonstrate how the method may be carried out in practice, certain embodiments will now be described with reference to the accompanying drawings above and the examples described below. The following examples are provided for illustration and do not limit the disclosure or scope of the claims and specification.

### Example 1

The samples used for this invention were obtained from a series of oil reservoirs located in Upper Austria. Three oil fields are classified as hot reservoirs with temperatures between 80-90° C., while one reservoir is stable at around 52° C. due to the shallower depth. Temperatures in topside facilities ranged from 35 to 40° C. Water-oil mixture samples were obtained directly at the well head of the production wells, while the aqueous phase of produced fluids from separator and buffer tanks were sampled.

In the examples below, the presence of three organic acids at proportions higher than 0.8% with the detection of four to seven lipids can be used to accurately predict the occurrence of souring.

Specifically, the analysis was performed as follows:

The samples collected for organic acid analysis consisted of 1 L of water, either taken directly from a tank or separated from the oil. After which, they were alkalized using 50 ml of a saturated NaHCO3-solution. The organic acids were then extracted three times with 300 ml of chloroform. Subsequently, the aqueous phase was acidified using 5 ml HCl (12 M) and extracted with 300 ml of ethyl acetate. The organic phases were combined, dried over sodium sulfate and evaporated to dryness under a stream of nitrogen. The extract was re-dissolved in 1 ml of diethyl ether for derivatization. Phenylsuccinic acid (100 μg) was added as an internal standard, to allow quantification of the detected acids.

Half of the organic acid extract was derivatized by methylation using diazomethane and analyzed by GC-MS. The other half was taken to derivatize succinic acids to succinimides using (R)-1-phenylethanamine. For a detailed description of the latter derivatization technique, see Jarling et al. (2015).

Medium-pressure liquid chromatography was performed according to the procedure described by Radke et al. (1980). The crude oil sample (30 mg) was diluted in 600 μl n-hexane and injected. Hetero compounds were retained on the pre-column. The aliphatic hydrocarbons were eluted with 40 ml n-hexane (flow rate: 8 ml/min). After an inversion of the flow direction, the aromatic hydrocarbons were eluted with 72 ml n-hexane (flow rate: 8 ml/min) from the main column. All fractions were vaporized and dried under a stream of nitrogen.

The gas chromatographic-mass spectrometric (GC-MS) analysis was performed on a gas chromatograph equipped with a fused silica capillary column (50 m length; 0.22 mm inner diameter; 0.25 μm film thickness, SGE Analytical Science). A sample volume of 1 μl was injected into the Programmed Temperature Vaporization (PTV) injector in splitless mode. The injector was programmed with a heating rate of 10° C./s from 50° C. to 300° C. Helium was used as a carrier gas with a flow rate of 1 ml/min. The GC oven was programmed to ramp from 50° C. (1 min hold time) to 310° C. (held for 30 min) with a heating rate of 3° C./min. The ion source temperature of the mass spectrometer was 230° C. and the ionization was performed by EI (electron impact) with an ionization energy of 70 eV. Mass spectra were recorded over a range of m/z 50-600 (derivatized organic acids of water extracts) and m/z 50-310 (aromatic hydrocarbon fraction) at a rate of 2.5 scans/s.

The samples collected for lipid analysis consisted of oil-water mixtures from active producing wells which were sampled using 10 L jerry cans and allowed to separate into phases before further processing. Water from topside facilities (separator tanks, water buffer tanks, and injectors) was filled into 5 L sterilized glass bottles.

Directly after sampling, 2 L of each water sample was filtered to collect and concentrate microbial cells using a Satorius filtration system, with glassfibre prefilters in combination 0.2 μm, I.D.: 50 mm filters. The filters containing the microbial cells were treated using a modification of the extraction method described by Bligh and Dyer (1959) to extract intact phospholipids. The filters containing the cells were saturated three times with a solvent mixture of methanol-dichloromethane-ammonium acetate buffer (10 mmol*L−1), 2:1:0.8 (v/v) in a beaker and sonicated for 10 min. The extracts were combined by transfer to a separation funnel and spiked with 50 μg deuterated [D31]-palmitoyl lysophosphatidylcholine (LPC) as an internal standard. The composition of the solvent was changed to about 1:1:0.9 (v/v) by adding dichloromethane and ammonium acetate buffer to allow phase separation. After separation of the organic phase, the water phase was extracted twice with 20 ml dichloromethane. The organic phases were combined, concentrated using a TurboVap® and dried under a stream of nitrogen.

The extract was dissolved in 1 ml of chloroform-methanol, 9:1 (v/v) and applied to a silica gel column (1 g silica gel 63-200 μm) and a florisil column used in sequence and eluted in the following order: 20 ml chloroform (neutral polar fraction), 50 ml methyl formate with 0.025% pure acetic acid (free fatty acid fraction), 20 mL acetone (glycolipid fraction) and 25 ml methanol (phospholipid fraction, only the silica column) and 25 ml of methanol-water, 6:4 (v/v) (highly polar fraction). The latter step contributes to significant improvement in the recovery of highly polar phospholipids from the silica column (Zink K, 2004). The highly polar fraction was collected and mixed with dichloromethane and water to achieve a methanol-dichloromethane-water ratio of 1:1:09 (v/v). After separation of the organic phase, the water phase was extracted twice with 20 mL dichloromethane. The organic phases obtained from the highly polar fraction were combined with the phospholipid fraction, evaporated and dried under a nitrogen stream.

Half of the phospholipid-containing extract was used for trans-esterification to obtain PLFAs from the intact phospholipids using a method described in the literature (Müller et al., 1990). A 500 μl addition of Trimethylsulfonium hydroxide was combined with the extract and the sealed vial was kept at 70° C. for 2 h. A GC-MS analysis was performed using a system equipped with a BPX5 fused silica capillary column (50 m length; 0.22 mm inner diameter; 0.25 μm film thickness). A sample volume of 1 μl was injected into the Programmed Temperature Vaporization (PTV) injector operated in splitless mode. The injector was programmed to ramp from 50° C. to 300° C. (10 min hold time) at a heating rate of 10° C./s. Helium was used as the carrier gas at a flow rate of 1 ml/min. The GC temperature program commenced at 50° C. (held for 1 min) followed by a temperature increase at a rate of 3° C./min to 310° C. (held for 30 min). The ion source temperature of the mass spectrometer was 230° C. The ionization was conducted by electron impact (EI) using an ionization energy of 70 eV. Mass spectra were recorded over a range of m/z 50-600 at a rate of 2.5 scans/s. PLFAs were identified by comparison of the mass spectra and retention times of their methyl esters to reference standards.

### Example 2

GC-MS based profiling of lipids and organic acids from over 500 samples from oil fields and topside facilities located in Upper Austria was completed. Three oil wells, from two different oil fields, as well at three tanks and 1 separator were samples over a period of three days, quarterly, for a period of three years. Of these locations, two of the wells, 1 tank, and the separator were sour. Analysis of the lipid and organic acid data revealed that a greater diversity of lipids were present in ‘souring affected systems’ but dramatically absent in souring-free systems. Based on these findings, it was determined that a combination of lipids and organic acids could be used to detect soured wells, tanks, and separators.

Additional data were analyzed and summaries are presented in Tables 1 and 2, which demonstrate: When all three organic acids presented are present at 0.8% of the total acid content or greater, in combination with the presence of four or more of the indicted lipids, there is a heightened probability that a souring process is taking place or will take place in the future. Based on samples collected over three years from the four sour sites in the field, analysis shows 100% correlation with souring.

Tables 1 and 2 list lipid and organic acid data for sour and non-sour wells.

TABLE 1

| | Sour | | | | | Sour | | | | | Sour | | | | | Sour | | | | |
|---|---|---|---|---|---|---|---|---|---|---|---|---|---|---|---|---|---|---|---|---|
| | Well 1 | | | | | Well 2 | | | | | Separator 1 | | | | | Tank 1 | | | | |
| PLFA | A | B | C | D | E | A | B | C | D | E | A | B | C | D | E | A | B | C | D | E |
| n-C₈-C₂₀ | + | + | + | + | + | + | + | + | + | + | + | + | + | + | + | + | + | + | + | + |
| n-C₂₁-C₂₄ | | | | | | + | + | | | + | | | + | | | | + | + | | |
| i-C₁₃ * | | | + | + | | + | + | | + | + | | + | + | + | | | + | + | | + |
| i-C₁₄ | | | | + | + | + | | | | + | + | + | + | + | + | + | + | + | | + |
| i-C₁₅ * | + | + | + | + | + | + | | + | + | + | + | + | + | + | + | + | + | + | + | + |
| i-C₁₆ | + | + | + | + | + | + | | + | + | + | + | + | + | + | + | + | + | + | + | + |
| i-C₁₇ * | + | + | + | + | + | + | | | | + | + | + | + | + | + | + | + | + | + | + |
| i-C₁₈ * | + | + | | + | | + | + | | + | | | + | + | + | | | | + | + | |
| i-C₁₉ | | | | | | − | | | | | | | + | | | | | + | | |
| ai-C₁₃ * | | | + | | | + | + | + | | | | | + | | + | | | + | | |
| ai-C₁₄ | | | + | | | + | | | | | | | + | | + | | | + | | + |
| ai-C₁₅ * | | | + | + | + | + | + | + | | + | + | + | + | + | + | + | + | + | + | + |
| ai-C₁₆ | | | + | + | + | + | | | | + | + | + | + | + | + | + | + | + | + | + |
| ai-C₁₇ * | + | + | + | + | + | + | + | + | + | + | + | + | + | + | | + | + | + | + | + |
| ai-C₁₈ | | | | | | | | | | | | | + | | | | + | | | |
| ai-C₁₉ | | | | | | | | | | | | | + | | | | | + | | |
| 10-Me16:0 | | | | | | | | | | | | | + | + | | | | + | + | + |
| 16:1ω7cis | | | | + | + | + | | | | + | + | + | + | + | + | + | + | + | + | + |
| 16:1ω7trans | | | | | | | | | | | + | | | | + | + | | | | + |
| 16:1ω9 | | | | | + | + | | | | + | | | + | | | | + | | | + |
| 17:1ω6 | | | | | | + | | | | + | + | | + | | | + | | | | |
| 17:1cyclo | | | | | | | | | | | + | + | + | | | + | | + | + | |
| 18:1ω7cis | | | | + | | + | + | + | + | + | + | + | + | + | + | + | + | + | + | + |
| 18:1ω7trans | | | | | | + | + | + | | | + | | | | | + | | | | |
| 18:1ω9 | + | + | + | + | | + | + | + | + | + | + | + | + | + | + | + | + | + | + | |

| | | |
|---|---|---|
| Non-sour | Non-sour | Non-Sour |
| Tank 2 | Tank 3 | Well 3 |

| PLFA | A | B | C | D | E | A | B | C | D | E | A | B | C | D | E |
|---|---|---|---|---|---|---|---|---|---|---|---|---|---|---|---|
| n-C₈-C₂₀ | + | + | + | + | + | + | + | + | + | + | + | + | + | + | + |
| n-C₂₁-C₂₄ | | | + | | | | | + | | | | | | | |
| i-C₁₃ * | | | | | | | | | | | | | | | |
| i-C₁₄ | | | | | | | | | | | | | | | |
| i-C₁₅ * | | | | | | | | | | | | | | | |
| i-C₁₆ | | | | | | | | | | | | | | | |
| i-C₁₇ * | | | | | | | | | | | | | | | |
| i-C₁₈ * | | | | | | | | | | | | | | | |
| i-C₁₉ | | | | | | | | | | | | | | | |
| ai-C₁₃ * | | | | | | | | | | | | | | | |
| ai-C₁₄ | | | | | | | | | | | | | | | |
| ai-C₁₅ * | | | | | | | | | | | | | | | |
| ai-C₁₆ | | | + | + | | | + | + | | | | | | | |
| ai-C₁₇ * | | | | | | | | | | | | | | | |
| ai-C₁₈ | | | | | | | | | | | | | | | |
| ai-C₁₉ | | | | | | | | | | | | | | | |
| 10-Me16:0 | | | | | | | | | | | | | | | |
| 16:1ω7cis | | | | | | | | | | | | | | | |
| 16:1ω7trans | | | | | | | | | | | | | | | |
| 16:1ω9 | | | | | | | | | | | | | | | |
| 17:1ω6 | | | | | | | | | | | | | | | |
| 17:1cyclo | | | | | | | | | | | | | | | |
| 18:1ω7cis | | | | | | | | | | | | | | | + |
| 18:1ω7trans | | | | | | | | | | | | | | | + |
| 18:1ω9 | | | + | + | | | | | + | | | | | + | |

Table outlining the detected lipids in the different samples. The (+) indicates that the lipid was detected in the sample, the lipids with an asterisk (*) can be used as indicators of souring. The letters below the site names indicate samples collected over time.

TABLE 2

| | Sour | | Sour | | Sour | | Sour | | Non-sour | | Non-sour | | Non-sour | |
|---|---|---|---|---|---|---|---|---|---|---|---|---|---|---|
| | | % of | | % of | | % of | | % of | | % of | | % of | | % of |
| Organic Acids | Well 1 | Total | Well 2 | Total | Sep. 1 | Total | Tank 1 | Total | Tank 2 | Total | Tank 3 | Total | Well 3 | Total |
| Benzoic acid | 8.84E+07 | 5.19% | 1.22E+06 | 0.70% | 3.98E+07 | 5.34% | 1.18E+08 | 18.14% | 1.07E+05 | 28.93% | 5.32E+05 | 28.35% | 1.52E+07 | 30.82% |
| Phenylacetic acid | 0.00E+00 | 0.00% | 0.00E+00 | 0.00% | 0.00E+00 | 0.00% | 0.00E+00 | 0.00% | 0.00E+00 | 0.00% | 7.01E+04 | 3.74% | 0.00E+00 | 0.00% |
| 2-Methylbenzoic acid | 2.45E+07 | 1.44% | 1.76E+05 | 0.10% | 1.17E+07 | 1.57% | 1.37E+07 | 2.12% | 0.00E+00 | 0.00% | 3.22E+04 | 1.72% | 0.00E+00 | 0.00% |
| 3-Methylbenzoic acid | 1.71E+08 | 10.03% | 1.56E+06 | 0.89% | 1.27E+08 | 17.04% | 1.52E+08 | 23.45% | 8.17E+04 | 22.17% | 0.00E+00 | 0.00% | 6.53E+06 | 13.27% |
| 2-Phenylpropionic acid | 0.00E+00 | 0.00% | 0.00E+00 | 0.00% | 0.00E+00 | 0.00% | 0.00E+00 | 0.00% | 0.00E+00 | 0.00% | 2.52E+05 | 13.42% | 0.00E+00 | 0.00% |
| 4-Methylbenzoic acid | 5.78E+07 | 3.39% | 5.56E+05 | 0.32% | 4.06E+07 | 5.44% | 4.72E+07 | 7.27% | 0.00E+00 | 0.00% | 3.07E+04 | 1.64% | 1.54E+06 | 3.14% |
| 2-Ethylbenzoic acid | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| o-Tolylacetic acid | 5.54E+06 | 0.33% | 0.00E+00 | 0.00% | 0.00E+00 | 0.00% | 6.20E+06 | 0.96% | 0.00E+00 | 0.00% | 2.87E+04 | 1.53% | 0.00E+00 | 0.00% |
| m-Tolylacetic acid | 2.47E+06 | 0.15% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| 3-Phenylpropionic acid | 0.00E+00 | 0.00% | 0.00E+00 | 0.00% | 0.00E+00 | 0.00% | 0.00E+00 | 0.00% | 0.00E+00 | 0.00% | 3.76E+04 | 2.00% | 0.00E+00 | 0.00% |
| p-Tolylacetic acid | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| 2,5-Dimethylbenzoic acid | 6.60E+06 | 0.39% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| 2,4-Dimethylbenzoic acid | 3.36E+06 | 0.20% | 1.29E+05 | 0.07% | 0.00E+00 | 0.00% | 0.00E+00 | 0.00% | 0.00E+00 | 0.00% | 0.00E+00 | 0.00% | 0.00E+00 | 0.00% |
| 3-Ethylbenzoic acid * | 1.50E+07 | 0.88% | 1.60E+07 | 9.13% | 1.14E+07 | 1.52% | 1.21E+07 | 1.86% | 0.00E+00 | 0.00% | 0.00E+00 | 0.00% | 9.21E+05 | 1.87% |
| 2,3-Dimethylbenzoic acid | 1.06E+07 | 0.62% | 9.98E+07 | 56.97% | 9.04E+06 | 1.21% | 8.94E+06 | 1.38% | 0.00E+00 | 0.00% | 0.00E+00 | 0.00% | 0.00E+00 | 0.00% |
| 3,5-Dimethylbenzoic acid * | 1.45E+07 | 0.85% | 4.02E+07 | 22.96% | 3.35E+07 | 4.48% | 3.36E+07 | 5.19% | 0.00E+00 | 0.00% | 5.90E+04 | 3.15% | 0.00E+00 | 0.00% |
| 3,4-Dimethylbenzoic acid * | 7.78E+07 | 4.56% | 1.51E+07 | 8.64% | 4.79E+07 | 6.42% | 4.75E+07 | 7.33% | 5.29E+04 | 14.34% | 0.00E+00 | 0.00% | 0.00E+00 | 0.00% |
| 1-Naphthoic acid | 7.31E+06 | 0.43% | 5.27E+04 | 0.03% | 1.04E+07 | 1.39% | 9.36E+06 | 1.44% | 0.00E+00 | 0.00% | 0.00E+00 | 0.00% | 0.00E+00 | 0.00% |
| 2-Naphthoic acid | 3.97E+07 | 2.33% | 3.23E+05 | 0.18% | 4.63E+07 | 6.21% | 4.48E+07 | 6.91% | 0.00E+00 | 0.00% | 0.00E+00 | 0.00% | 8.55E+05 | 1.74% |
| Cyclohexanecarboxylic acid | 0.00E+00 | 0.00% | 0.00E+00 | 0.00% | 0.00E+00 | 0.00% | 0.00E+00 | 0.00% | 0.00E+00 | 0.00% | 4.80E+05 | 25.57% | 0.00E+00 | 0.00% |
| 2-Methylcyclohexanecarboxylic acid | 0.00E+00 | 0.00% | 0.00E+00 | 0.00% | 0.00E+00 | 0.00% | 0.00E+00 | 0.00% | 0.00E+00 | 0.00% | 4.46E+04 | 2.38% | 0.00E+00 | 0.00% |
| 3-Methylcyclohexanecarboxylic acid | 0.00E+00 | 0.00% | 0.00E+00 | 0.00% | 0.00E+00 | 0.00% | 0.00E+00 | 0.00% | 0.00E+00 | 0.00% | 2.05E+05 | 10.91% | 0.00E+00 | 0.00% |
| 4-Methylcyclohexanecarboxylic acid | 0.00E+00 | 0.00% | 0.00E+00 | 0.00% | 0.00E+00 | 0.00% | 0.00E+00 | 0.00% | 0.00E+00 | 0.00% | 5.50E+04 | 2.93% | 0.00E+00 | 0.00% |
| Cyclohexaneacetic acid | 0.00E+00 | 0.00% | 0.00E+00 | 0.00% | 0.00E+00 | 0.00% | 0.00E+00 | 0.00% | 0.00E+00 | 0.00% | 4.99E+04 | 2.66% | 0.00E+00 | 0.00% |
| | | Percent | | Percent | | Percent | | Percent | | Percent | | Percent | | Percent |
| | | of | | of | | of | | of | | of | | of | | of |
| Organic Acids | Well 1 | Total | Well 2 | Total | Sep. 1 | Total | Tank 1 | Total | Tank 2 | Total | Tank 3 | Total | Well 3 | Total |
| n-C4 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C6 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C8 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C9 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C10 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C11 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C12 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C14 | 0.00E+00 | 0.00% | 0.00E+00 | 0.00% | 0.00E+00 | 0.00% | 4.31E+06 | 0.66% | 0.00E+00 | 0.00% | 0.00E+00 | 0.00% | 0.00E+00 | 0.00% |
| n-C15 | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-C16 | 0.00E+00 | 0.00% | 0.00E+00 | 0.00% | 0.00E+00 | 0.00% | 6.45E+06 | 1.00% | 7.69E+04 | 20.85% | 0.00E+00 | 0.00% | 0.00E+00 | 0.00% |
| n-C18 | 0.00E+00 | 0.00% | 0.00E+00 | 0.00% | 0.00E+00 | 0.00% | 5.82E+06 | 0.90% | 5.05E+04 | 13.71% | 0.00E+00 | 0.00% | 0.00E+00 | 0.00% |
| Methylsuccinic acid | 8.81E+08 | 51.70% | 0.00E+00 | 0.00% | 2.45E+08 | 32.87% | 0.00E+00 | 0.00% | 0.00E+00 | 0.00% | 0.00E+00 | 0.00% | 0.00E+00 | 0.00% |
| Dimethylsuccinic acid | 1.90E+08 | 11.13% | 0.00E+00 | 0.00% | 8.74E+07 | 11.72% | 1.00E+08 | 15.46% | 0.00E+00 | 0.00% | 0.00E+00 | 0.00% | 1.51E+07 | 30.60% |
| Ethylsuccinic acid | 8.44E+07 | 4.95% | 0.00E+00 | 0.00% | 3.57E+07 | 4.79% | 3.84E+07 | 5.93% | 0.00E+00 | 0.00% | 0.00E+00 | 0.00% | 8.27E+06 | 16.80% |
| Isopropylsuccinic acid | 8.58E+06 | 0.50% | 0.00E+00 | 0.00% | 0.00E+00 | 0.00% | 0.00E+00 | 0.00% | 0.00E+00 | 0.00% | 0.00E+00 | 0.00% | 0.00E+00 | 0.00% |
| n-Propylsuccinc acid | 1.59E+07 | 0.93% | 0.00E+00 | 0.00% | 0.00E+00 | 0.00% | 0.00E+00 | 0.00% | 0.00E+00 | 0.00% | 0.00E+00 | 0.00% | 8.70E+05 | 1.77% |

Table outlining the detected organic acids in the different samples. The numbers are peak areas of each organic acid in the sample, and the percentages indicate percent abundances for each acid in relation to total acids for that sample. The names with an asterisk (*) indicate the organic acids that can be used as indicators, when present at abundances of 0.8% or more in the sample

What is claimed is:

1. A method for the early detection of souring comprising:
i) preparing an aqueous sample comprising microorganisms; and,
ii) analyzing the sample for the presence of lipids and organic acids selected from the group consisting of iso- and anteiso-methyl branched fatty acids containing 13, 15, or 17 carbons, iso-methyl branched fatty acids containing 18 carbons, 3,4-dimethylbenzoic acid, 3,5-dimethylbenzoic acid, and 3-ethylbenzoic acid;
wherein the presence of the combination of one or more of 3,4-dimethylbenzoic acid, 3,5-dimethylbenzoic acid, and 3-ethylbenzoic acid at a concentration greater than or equal to 0.8% combined with four or more lipids indicates the presence of souring.

2. The method of claim 1 wherein each of 3,4-dimethylbenzoic acid, 3,5-dimethylbenzoic acid, and 3-ethylbenzoic acid are combined in the sample and the total concentration of the combination is present at a concentration greater than or equal to 0.8%.

3. The method of claim 1 wherein each of 3,4-dimethylbenzoic acid, 3,5-dimethylbenzoic acid, and 3-ethylbenzoic acid are combined in the sample and each of 3,4-dimethylbenzoic acid, 3,5-dimethylbenzoic acid, and 3-ethylbenzoic acid is present at a concentration greater than or equal to 0.8%.

4. The method according to claim 1, wherein the analyzing step comprises a quantitative or qualitative detection of lipids and organic acids.

5. The method according to claim 1, further comprising detecting five of the lipids.

6. The method according to claim 1, further comprising detecting at least six of the lipids.

7. The method according to claim 1, further comprising detecting all seven of the lipids.

8. The method according to claim 1, wherein the analyzing comprises contacting the sample with a reagent composition or test device that enables qualitative or quantitative detection of lipids and organic acids that are characteristic of said compounds.

9. A method for treating a souring aqueous system comprising:
i.) detecting souring according to the method of claim 1; and
ii.) applying a biocide, hydrogen sulfide scavenger, or combination thereof.
